(19) United States
(12) Patent Application Publication
(10) Pub. No.: US 2006/0277612 A1
(43) Pub. Date: Dec. 7, 2006

(54) Methods and compositions for defining gene function

(76) Inventors: Daniel L. Small, The Woodlands, TX (US); Arthur T. Sands, The Woodlands, TX (US)

Correspondence Address: LEXICON GENETICS INCORPORATED, 8800 TECHNOLOGY FOREST PLACE, THE WOODLANDS, TX, 77381-1160, US

(21) Appl. No.: 11/441,837

(22) Filed: May 26, 2006

Related U.S. Application Data
(60) Provisional application No. 60/684,927, filed on May 26, 2005.

(51) Int. Cl. A01K 67/027; C12Q 1/68

(52) U.S. Cl. 800/3; 435/6; 800/18

(57) ABSTRACT

The present invention relates to a process for the efficient and practical production and analysis of mutated mouse ES cell clones useful for defining the physiological role of genetically encoded biopolymers and for the in vivo testing for enhanced resistance or sensitivity to injury challenge or other physiological insult.

The present application claims the benefit of U.S. Provisional Application Ser. No. 60/684,927, which was filed on May 26, 2005, and is herein incorporated by reference in its entirety.

### 1.0. FIELD OF THE INVENTION

The present invention relates to the development of a process for systematically screening the mammalian genome for genes encoding products that modulate cellular homeostasis.

### 2.0. BACKGROUND OF THE INVENTION

Most human therapeutics directly or indirectly interact with products encoded by the human genome. Consequently, scientific scrutiny has turned to defining that portion of the human genome coding sequence that presents a clear opportunity for medical intervention. Many therapeutic products act by modulating physiology. Of the thousands of biochemically and structurally related products encoded within the human genome, the scientific community has defined the physiological roles of only a fraction of these products.

### 3.0. SUMMARY OF THE INVENTION

The present invention relates to a process for identifying genes encoding products modulating healing and repair, or the impairment thereof, that individually subjects a collection of at least about 40 distinct genetically engineered knockout mouse lines to an injury challenge and measures their resistance, recovery, or sensitivity to this challenge by assessing any of several biological markers in vivo.

In certain embodiments, the injury challenge is by ionizing radiation, which, among other things, allows for reproducible delivery of desired dosages and can be targeted to specific tissues and areas of the body (by aiming the beam or employing appropriate shielding). In certain additional embodiments, the injury challenge is by a chemotherapeutic agent, traumatic or ischemic injury, introduction into the body of exogenous products, or by chemical mutagenesis as well as high energy particle beams or light, and additional forms of radiation induced injury.

In certain embodiments, the injury challenge can be above the normal LD50 for wildtype test animals, and in certain additional embodiments, the injury challenge is at a sublethal dose.

Certain embodiments of the described invention relate to the observation that brain injury (from any of a wide variety of means) can increase the incidence of Alzheimer's disease. Accordingly, genes/proteins that reduce the recovery time after brain or head injury are useful in the treatment and prevention of symptoms relating to neuropathologies associated with Alzheimer's disease, Parkinson's, and other neurodegenerative disorders as well as retinopathy, macular degeneration, corneal lesions, and retinal degeneration.

### 4.0. DESCRIPTION OF THE SEQUENCE LISTING

The Sequence Listing provides the sequences of several oligonucleotide primers useful in certain specific embodiments of the present invention.

### 5.0 DETAILED DESCRIPTION OF THE INVENTION

The mouse is a tractable and predictive model organism for characterizing the physiological function of human biological sequences. As a mammal, the mouse shares many, if not all, of the major organ systems of humans and often modulates the functions of these organ systems using orthologous products. Furthermore, the mouse also allows for the genetic engineering of its genome since mouse embryonic stem (ES) cell technology provides an avenue for chromosome engineering and, consequently, the direct testing of genomic hypotheses.

Thus, certain embodiments of the present invention describe processes for producing a collection of individually characterized mutated mouse ES cell clones using established methods, (see generally U.S. Pat. Nos. 6,136,566, 6,080,576, 6,436,707, 6,204,061, 6,653,113, 6,689,610, 5,487,992, 5,464,764, and 5,789,215 all of which are herein incorporated by reference in their entirety), and using the clones to generate mice capable of germline transmission of the genetically engineered mutated alleles. Where these mice contain mutated alleles that effectively ablate the functional expression of the mutated allele, these animals are often referred to as “Knockout” mice.

One use for genetically engineered animals is to produce offspring that are homozygous or heterozygous for the mutated allele, or offspring that are normal (wildtype) by virtue of not incorporating the mutated allele in their genome. In certain embodiments, the offspring (or progeny thereof) are subject to physiological injury challenge (e.g., irradiation, hypo or hyperthermia, chemotherapy, mutagenic challenge, hypo or hyperoxygenation, or agents or other stimuli that induce apoptotic cell death), and then subject to diagnostic tests to reveal whether the presence of the mutated allele (in heterozygous or homozygous states) confers enhanced resistance or sensitivity to physiological challenge. In certain embodiments, age and/or gender matched cohorts of wildtype animals, or animals heterozygous or homozygous for the mutated allele are subject to physiological injury challenge. In certain additional embodiments, genetically matched cohorts are sorted by age and subject to injury challenge to assess whether the physiological role of the gene changes as a function of age. In certain embodiments, knockout animals produced according to the described methods are systematically subject to carcinogenic challenge to assess whether the engineered mutation renders affected cells and/or animals more susceptible to carcinogenic challenge (indicating that the mutated gene encodes a tumor suppresser) or less susceptible to injury challenge (which indicates that antagonizing the wildtype product encoded by the unaltered from of the mutated locus would suppress the progression of cancer).

In certain embodiments, tissues are selectively subject to physiological or injury challenge. In such cases, such as in the case with neural tissue that can be targeted by aiming radiation at areas of the body rich in neural tissue such as the brain and spine, neuroprotective or neurosensitizing genes/products can be identified that can be targeted or exploited to either protect neural tissue from death or damage (i.e., to prevent loss of neural function or paralysis), or, alternatively, to render certain neural tissues more sensitive to apoptotic stimuli (as might be desired for brain or neural tumors). Depending on the nature of the injury challenge, the neuroprotective cells and/or processes can be targeted by the injury challenge, or present in areas of the body that are not directly targeted by the injury challenge (such as in instances where circulatory cells present throughout the body subsequently home to areas of the body that have been targeted and damaged by, for example, beams of radiation).

In certain embodiments, the described analysis is conducted by exposing cells or animals containing the described genetically engineered alleles in either a heterozygous or homozygous state to dose or net doses of, for example, radiation mediated injury challenge (along with wildtype controls). In certain embodiments, additional forms of mutagenic injury challenge can include, but are not limited to, chemical mutagens (see generally “Dangerous Properties of Industrial Materials”, 7th Ed., by N. Irving Sax and Richard J. Lewis for a partial list of potential chemical mutagens, and Cancer Chemotherapy and Biotherapy: Principles and Practice, Chabner and Longo (Eds), Third Edition, Lippincott Williams and Wilkins, August 2001 both of which are herein incorporated by reference in their entirety), ultraviolet light, and other forms of radiation.

In certain embodiments, injury challenge is made using ionizing radiation such as, but not limited to, gamma rays, x-rays, high energy x-rays, electron beams, radon gas, and other forms of radiation suitable for such applications (for additional sources/examples see “Textbook of Radiation Oncology,” S. A. Leibel et al., eds., W. B. Saunders, 1998, ISBN: 0721653367, herein incorporated by reference in its entirety).

In certain embodiments, the genetic screen by physiological injury challenge will comprise at least about 20, 40, 100, 200, 300, 500, 1,000, 5,000, 10,000, 20,000, or 25,000 different mutated alleles (for additional sources of such alleles, see the methods and collections described in U.S. Pat. Nos. 6,080,576, 6,436,707, 6,136,566, and 6,207,371 each of which is herein incorporated by reference in its entirety).

Certain embodiments of the present invention relate to the discovery that certain subsets of neuronal cells can be used as proxies for an animal's, or animal line's in the case of genetically engineered animals, propensity for neurodegeneration or relative resistance to neurodegeneration in the body. Examples of such neuronal cells include newly replicated neurons and/or cells having increased rates of cell division relative to mature neurons. In certain embodiments, Ki67 positive cells in the dentate gyrus of the hippocampus can be readily identified/quantified by traditional means. Although Ki67 expression provides a facile and quantitative measure of the desired neuronal cell populations, additional markers can be used to verify that numbers of Ki67 positive cells after injury challenge are present in the desired neuronal tissues and cells. Examples of such additional neuronal markers include, but are not limited to, double cortin (DCX), class III beta-tubulin (TuJ1), and polysialylated neural cell adhesion molecule (PSA-NCAM).

The present invention is further illustrated by the following examples, which are not intended to be limiting in any way whatsoever.

### 6.0. EXAMPLES

### 6.1. Production of Gene Trapped ES Cells

Gene trapping is a method of random insertional mutagenesis that uses a fragment of DNA (in some cases introduced via a retroviral RNA proxy) coding for a reporter or selectable marker gene as a mutagen. In certain embodiments, gene trap vectors have been designed to integrate into introns or exons in a manner that allows the cellular splicing machinery to splice vector encoded exons to contranscribed endogenous exons. In certain embodiments, gene trap vectors contain selectable marker sequences that are preceded by strong splice acceptor sequences and are not preceded by a promoter. In certain embodiments, when such vectors integrate into a gene, the cellular splicing machinery splices exons from the trapped gene onto the 5′ end of the selectable marker sequence. In certain embodiments, such selectable marker genes can only be expressed if the vector encoding the gene has integrated into an intron. The resulting gene trap events are subsequently identified by selecting for cells that can survive selective culture. Alternatively, stably transduced cells can be selected by identifying whether they incorporate the gene trap vector (by, for example, screening by PCR or inverse PCR) or express a chromogenic or enzymatic marker (luciferase, fluorescence, enzyme activity on a chromogenic substrate, etc.) sequence.

Embryonic stem cells (Lex-1 cells from derived murine strain A129) were mutated by infection with the retroviral gene trapping vector VICTR48 at an input ratio (or multiplicity of infection) of approximately 0.3. By using the low input ratio, approximately 95 percent of the ES clones that stably integrate the proviral form of the vector are predicted to contain a single integration event. After selecting for ES clones that stably incorporated the vector via G418 selection (although additional means of selecting such cells could have been used such as, for example, green fluorescent protein, luciferase, antibiotic resistance markers, etc.), the selected cells were seeded onto irradiated feeder cells (that stably express LIF) and cultured to confluence. The confluent wells were subsequently split 1-to-3 into three 96 well plates and again allowed to grow to confluence. Two of the resulting plates were cryogenically preserved and the third plate was processed essentially as follows:

The media was removed and the cells were rinsed twice with 100 μl PBS. 50 μl lysis solution (50 mM Tris pH 7.5, 50 mM EDTA pH 8.0, 100 mM NaCl, 1% SDS, and 2 mg/ml Proteinase K) was added to each well, the plate was sealed and incubated at 65 degrees C. overnight. 150 μls of 95% ethanol was added to each well and the plates were left standing at RT for 2 hours. Supernatant was aspirated and the wells were washed with 150 μl 70% ethanol. After the ethanol was aspirated, the wells were allowed to dry (approximately 2 hrs at room temperature). 200 μl TE was added to each well and left in at RT overnight to gently rehydrate the genomic DNA. 10 μl of genomic DNA was then transferred, in triplicate, to new 96-well plates for endonuclease digestion and sequence analysis.

Alternatively, in certain embodiments the gene trapped clones are subject to 5′ RACE and the resulting cDNA fragments sequenced by, for example, cycle sequencing.

### 6.2. Screening Mutated Animals for Altered Response to Induced Neurodegeneration Challenge

Injury challenge can induce neurodegeneration in animals. Of the various forms of injury challenge, radiation can be targeted to selected tissues and cells. Targeting gamma irradiation to new neuronal cells in the brain can induce delayed neuronal death in this cell population. Comparing the extent of neurodegeneration in this cell population in mutant and wild-type mice provides insight into whether the absence of the protein normally encoded by the gene in its non-mutated state modulates neurodegeneration. Applicants have identified a subset of the neuronal cell population in the dentate gyrus of the hippocampus that contains newly formed neurons. These newly formed neurons are generally not post-mitotic like most other neurons in the brain and therefore remain more sensitive to the radiation challenge. Applicants have also determined that the immunohistochemical marker Ki67 can be used to quantify the number of non-postmitotic neurons remaining after radioactive challenge, and that the number of Ki67 positive neurons is a predictor of neurodegenerative disease progression. Accordingly, the described methods are useful for identifying genes that play a role in preventing or enhancing neurodegeneration. These genes represent viable targets for the therapeutic intervention in stroke, Alzheimer's disease, Huntington's, Parkinson's, and traumatic brain injury.

Typically, each animal is treated by head only gamma-radiation. After receiving mice that have recovered from 3 Gy whole-body radiation (usually a period of about 3 weeks), the head of the mouse was exposed to an additional 5 Gy of head-only radiation followed by another 5 Gy of head-only radiation 3 days after the first exposure. Radiation was delivered using unanesthetized mice which were then placed in plastic cones to immobilize them, which were then placed in a 250 mL glass beaker wrapped in lead foil such that the heads of the animals remained protruding above the beaker and thus exposed to radiation. The mice were placed in the GAMMATOR G-50-B GAMMA IRRADIATOR ¹³⁷Cs source for about 2 minutes (5 Gy) and then returned to the cage. Although previously irradiated animals were used in the above studies, the presently described methods are equally applicable to testing animals that have not been previously subject to whole-body irradiation.

Six days after the first head-only exposure, the mice were euthanized by CO2 exposure and the brains were harvested and immersion fixed in 4% buffered formalin for 24 hrs. After being fixed, the brains were placed in a coronal brain matrix and trimmed (2 mm off the rostral and caudal portion of the brain are discarded) and cut in half at approximately Bregma +0.5. The caudal half of the brain was placed in a labeled processing cassette with a small section of intestine to serve as a positive control for Ki67 immunostaining and placed in phosphate buffered saline before processing.

Labeled cassettes containing the brains for histological evaluation were loaded into the baskets and placed in the retort of the Leica TP1050 tissue processor. The brains were dehydrated through a series of ethanol washes followed by xylene washes and were processed in paraffin. Using a Tissue-Tek embedding console, the brains were placed cut side down, on a pre-hardened base of paraffin and then the mold was filled with paraffin and hardened on the cold plate. The block was trimmed 1-1.5 mm with the microtome and then two 5 micron sections were cut and collected. The sections were floated on a heated (40-50° C.) water bath and mounted on microscope slides.

To perform Ki67 immunohistochemistry, the slides containing the brain slices were loaded into a Dako autostainer and endogenous biotin was blocked by treating with avidin and biotin each for 10 min, then blocked with 20% normal rabbit serum for 20 min. The sections were then treated with Dako primary anti-mouse Ki67 (1:30) for 1 hr, washed and then incubated with Vector biotinylated rabbit anti rat IgG (mouse absorbed) 1:400 for 1 hr. After rinsing, sections were then exposed to Vector Elite ABC (1:80) for 1 hr, rinsed and then Vector DAB for 5 min, rinsed and then counter-stained with Gill's hematoxylin, and mounted.

The number of Ki67 immunopositive cells in the granule cell area in the left and right dentate gyrus of the hippocampus of one-to-two 5 micron paraffin sections of brain were counted and averaged to represent the number of surviving new neurons following irradiation challenge. One observation made during these studies is that certain non-neuronal cells can also stain positive for Ki67, such as, but not limited to, immune and inflammatory cells. To assess the extent to which these cells may confound the desired conclusions, counter-screening with additional markers can be used to verify that the Ki67 positive cells are indeed of neural origin. Accordingly, certain embodiments of the present invention include a step where a test sample is stained with an additional neuronal marker.

### 6.2.1. Summary of Results

Three ‘benchmark’ mutant mouse strains (X-ray repair complementing defective in Chinese hamster 5 XRCC5, Ku80; xeroderma pigmentosum complementation group C, XPC; and p53) have been tested in the above brain irradiation challenge assay. These genes were selected because of their established roles in DNA damage repair (Ku80 and XPC) or apoptotic cell death (p53). The XPC mutant mice had significantly fewer Ki67-positive cells in the dentate gyrus after exposure to irradiation than their wildtype (wt) litter mates. The Ku80 mutant mice were so much more sensitive to the irradiation-induced damage that the exposure level had to be reduced to 2 Gy from 5 Gy to avoid a reduced viability following exposure. Even after only 200 rad irradiation, the Ku80 mice had significantly fewer Ki67-positive cells in the dentate gyrus. In contrast, the p53 mutant mice had significantly more Ki67-positive cells following the irradiation challenge than the wt mice consistent with the p53 mutant mice being resistant to the brain irradiation challenge.

In addition to the ‘benchmark’ strains described above, 805 mutant strains have been challenged in this assay as part of a broader phenotypic screen.

An example of a gene we have identified using this assay is the mouse ortholog of the human solute carrier family 35, member F5 (SLC35F5)—carbohydrate translocator, Accession:NM_{—}028787 Gene:1300003P13RIK PROTEIN. Mice homozygous for a mutation in this gene showed an increased resistance to the brain irradiation challenge and had more Ki67-positive cells remaining after being irradiated.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description. Such modifications are intended to fall within the scope of the appended claims. All cited publications, patents, and patent applications are herein incorporated by reference in their entirety in all ways that are consistent with and support the present disclosure.

What is claimed is:

1. A process for identifying genes involved in injury repair or enhanced sensitivity to injury, comprising:
a) subjecting a collection of at least about 40 distinct genetically engineered knockout mouse lines to an injury challenge; and
b) measuring the surviving Ki67 positive cell population.

2. The process of claim 1 wherein said injury challenge is by ionizing radiation.

3. The process of claim 1 wherein said injury challenge is by a chemotherapeutic agent.

4. The process of claim 1 wherein said injury challenge is at a sublethal dose.

5. A process for identifying genes encoding products that modulate neurodegeneration, comprising:
a) subjecting a mouse to a source of neuronal damage;
b) measuring the surviving Ki67 positive cell population; and
c) using an additional marker to screen for Ki67 positive cells that are not of neuronal lineage.

6. A process according to claim 5 wherein said additional marker is drawn from the group consisting of double cortin, class III beta tubulin, and polysialylated neural cell adhesion molecule.
